(19) United States
(12) Patent Application Publication
(10) Pub. No.: US 2007/0135353 A1
(43) Pub. Date: Jun. 14, 2007

(54) Crystalline n-formyl hydroxylamine compounds

(76) Inventors: Joel Slade, Flanders, NJ (US); Martin Muller, Basel (CH); Hui Liu, Green Brook, NJ (US); Joginder S. Bajwa, Elmwood Park, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/550,631

(22) PCT Filed: Apr. 1, 2004
(86) PCT No.: PCT/EP04/03478

§ 371 Date: Dec. 7, 2006

Related U.S. Application Data
(60) Provisional application No. 60/459,726, filed on Apr. 2, 2003.

(51) Int. Cl. A61K 38/04; C07K 5/08; A61K 31/426; A61K 31/421; A61K 31/19

(52) U.S. Cl. 514/19; 514/365; 514/374; 514/575; 514/423; 548/200; 548/530; 548/215

(57) ABSTRACT

Certain N-formyl hydroxylamine compounds, such as N-[1-oxo-2-alkyl-3-(N-hydroxyformamido)-propyl]-(carbonylamino-aryl or -heteroaryl)-azacyclo₄₋₇alkanes or thiazacyclo₄₋₇alkanes or imidazacyclo₄₋₇alkanes are useful in the treatment of bacterial infections. Disclosed are crystalline salts of such compounds of formula (I), wherein M is a mono- or di-valent metal; a is ½ or 1.

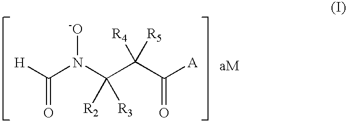

This invention is directed to crystalline N-formyl hydroxylamine compounds, to the uses of these compounds in various medicinal applications, including treating disorders amenable to treatment by peptidyl deformylase (PDF) inhibitors, such as treatment of bacterial infections, and to pharmaceutical compositions comprising these crystalline compounds.

PDF is a metallopeptidase found in prokaryotic organisms, such as bacteria. Protein synthesis in prokaryotic organisms begins with N-formyl methionine (fMet). After initiation of protein synthesis, the formyl group is removed by the enzyme PDF; this activity is essential for maturation of proteins. It has been shown that PDF is required for bacterial growth. See Chang et al., J. Bacteriol., Vol. 171, pp. 4071-4072 (1989); Meinnel et al., J. Bacteriol., Vol. 176, No. 23, pp. 7387-7390 (1994); and Mazel et al., EMBO J., Vol. 13, No. 4, pp. 914-923 (1994). Since protein synthesis in eukaryotic organisms does not depend on fMet for initiation, agents that will inhibit PDF are attractive candidates for development of new anti-microbial and anti-bacterial drugs.

Co-pending application Ser. No. 10/171,706, filed Jun. 14, 2002 (incorporated herein by reference in its entirety), PCT equivalent published as WO 02/102790 A1, discloses novel N-formyl hydroxylamine compounds that inhibit PDF and are therefore useful as antibacterial agents. Additionally, PCT application WO 99/39704 discloses other N-formyl hydroxylamine derivatives that are antibacterial agents by virtue of their PDF inhibiting capabilities. The compounds disclosed in these patent applications are amorphous, i.e., they are not crystalline.

For the formulation of drug compositions it is important for the drug substance to be in a form in which it can be conveniently handled and processed. Chemical stability (shelf-life) and purity are also important considerations for drug substances, such as antibiotics. Amorphous materials may present significant problems in this regard. For example, amorphous drug substances typically are difficult to formulate, provide for unreliable solubility, and are often found to be chemically unstable and unpure. As is clear to one skilled in the art, crystalline forms of such drug substances may solve or alleviate such problems. Thus it would be highly desirable to have crystalline forms of the antibacterial agents disclosed in WO 02/102790 and WO 99/39704.

The procedures and examples disclosed in WO 02/102790 and WO 99/39704 teach the formation of amorphous forms of the compounds disclosed therein. It has presently been discovered that crystalline salts of such compound can be obtained. Thus, the present invention is directed to crystalline salts of the compounds disclosed in WO 02/102790 and WO 99/39704, e.g., to a crystalline salt of formula (I): 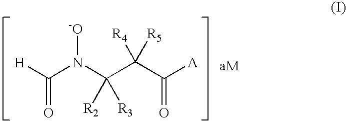
wherein

M is a mono- or di-valent metal;

a is ½ or 1;

each of R₂, R₃, R₄ and R₅, independently, is hydrogen or an aliphatic group, or (R₂ or R₃) and (R₄ or R₅), collectively, form a C₄-C₇cycloalkyl;

A is of the formula (Ia), (Ib), (Ic), (Id) or (Ie) 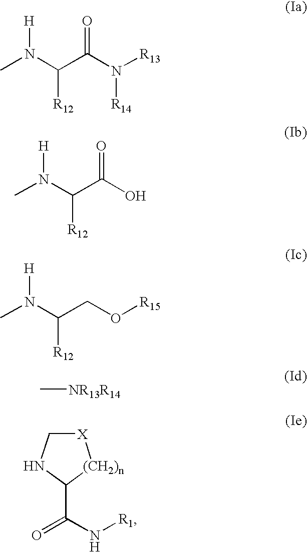
wherein

R₁₂ is the side-chain of a natural or a non-natural alpha amino acid;

R₁₃ and R₁₄, independently, represent hydrogen, or optionally substituted C₁-C₈alkyl, cycloalkyl, aryl, aryl(C₁-C₆alkyl), heterocyclic or heterocyclic(C₁-C₆alkyl);

R₁₅ is hydrogen, C₁-C₆alkyl or an acyl group;

X is —CH₂—, —S—, —CH(OH)—, —CH(OR)—, —CH(SH)—, —CH(SR)—, —CF₂—, —C═N(OR)— or —CH(F)—, wherein R is alkyl;

R₁ is aryl or heteroaryl; and

n is 0-3, provided that when n is 0, X is —CH₂—.

The compounds of the invention are in the form of solid crystalline salts. Preferably the crystalline salts are metal salts, preferably of divalent metals, although for some compounds it is possible to form crystalline solids by using monovalent counter ions, such as Na. The counter ion is preferably Mg, Ca or Zn.

The compounds of the invention are typically in the form of a hydrate or a mixed solvate/hydrate. Typically, the crystalline salt of the invention contains about 2 to 8 waters of hydration, more typically about 2 to 6 waters of hydration, and even more typically about 2 to 4 waters of hydration. Particularly preferred salts of the invention are the tetrahydrates. Thus, the crystalline salt of the invention typically comprises greater than 2% water, more typically about 4 to about 12% water and even more typically about 8 to about 9% water. Solvates may be of one or more organic solvents, such as lower alkyl alcohols, such as methanol, ethanol, isopropanol, butanol or mixtures thereof.

The present invention is also directed to a process for preparing the crystalline salts of the invention. The process of the invention comprises dissolution of the amorphous, non-salt form of the compound of formula (I) in a suitable solvent, contacting the dissolved compound with a base and with a metal salt, under conditions suitable to form the desired crystalline salt of formula (I). The base can be added first, the metal salt first, or both can be added simultaneously. The base is preferably in the form of an aqueous solution of an alkaline metal hydroxide, such as KOH or NaOH. The amount of base is sufficient to achieve a pH of about 8 to about 11, preferably about 8.5 to about 9.5. The metal salt can be inorganic or organic; however, it must be soluble in, i.e., dissociate in, the reaction medium. The metal salt is preferably a salt of a divalent cation, e.g., Mg, Ca or Zn. The anion of the metal salt can be, e.g., chloride, sulphate, acetate, 2-ethylhexanoate, and the like. By contacting non-salt form of the compound of formula (I) with the alkaline metal hydroxide, a salt of the compound of formula (I) is formed (first salt) with the counter ion (cation) being the metal from the alkaline metal hydroxide, e.g., Na or K. The cation of the metal salt then displaces the metal of the first salt to form the crystalline salt of the invention (second salt). The suitable solvent is preferably water, but it can also be one or more organic solvents, such as lower alkyl alcohols, such as methanol, ethanol, isopropanol or mixtures thereof. The temperature for this process is not known to be critical and can vary from about 20° C. to about 60° C., preferably about 30° C. to about 50° C. The reaction time is typically about 1 hour to about 6 hours, preferably about 3 hours to about 4 hours. Typically the process is performed under agitation. The crystalline salt can then be isolated, dried, and/or purified by conventional techniques known in the art, e.g., filtration, re-crystallization, drying under vacuum and the like.

It is possible to prepare the crystalline monovalent metal salt of some of the compounds of formula (I). To prepare a monovavent metal salt, e.g., the sodium salt, the non-salt form of the compound of formula (I) is dissolved in a suitable solvent, preferably water or an alcohol such as methanol, ethanol or iso-propanol, optionally including water, and the dissolved compound is contacted with a monovalent metal hydroxide, e.g., NaOH or KOH, under conditions suitable for formation to the monovalent metal salt, e.g., the sodium salt. The salt thus formed will be in solution which must be further manipulated to make the crystalline salt of the invention, e.g., the solvent can be removed, e.g., via vacuum distillation, or an anti-solvent can be added to cause the desired crystalline salt of the invention to precipitate. Such anti-solvents must miscible with the solvent in use, but the compound will be substantially insoluble in the anti-solvent. Typical examples of anti-solvents include acetone and lower alkyl alcohols, such as methanol, ethanol, isopropanol and the like. Preferably the monovalent metal hydroxide is in an aqueous solution. Other conditions are the same or similar to those described in the preceding paragraph.

The crystalline salts of the invention can be analyzed by use of standard X-ray powder diffraction techniques known in the art. Some preferred compounds of the invention are wherein the X-ray powder diffraction pattern comprises crystalline peaks with 2-theta angles (Cu—K_{α} radiation) at least five of the following positions (preferably at least 6, more preferably at least 7, more preferably at least 8, more preferably at least 9, more preferably at least 10 and most preferably all 11): 6.8±0.1, 13.7±0.1, 12.2±0.1, 14.5±0.1, 15.2±0.1, 18.1±0.1, 20.6±0.1, 22.0±0.1, 22.4±0.1, 24.5±0.1 and 30.9±0.1. Typically, the analysis is carried out at 50% relative humidity.

Preferably, the present invention provides crystalline salts of N-[1-oxo-2-alkyl-3-(N-hydroxyformamido)-propyl]-(carbonylamino-aryl or -heteroaryl)-azacyclo₄₋₇alkane or thiazacyclo₄₋₇alkanes or imidazacyclo₄₋₇alkanes. In one embodiment, A is of formula (Ie) and R₁ is a heteroaryl of formula (II) 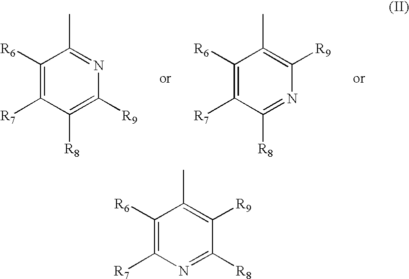
wherein each of R₆, R₇, R₈ and R₉, independently, is hydrogen, alkyl, substituted alkyl, hydroxy, alkoxy, acyl, acyloxy, SCN, halogen, cyano, nitro, thioalkoxy, phenyl, heteroalkylaryl, alkylsulfonyl or formyl.

In another embodiment, A is of formula (Ie) and R₁ is preferably a heteroaryl of formula (II.1) 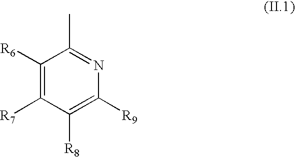
wherein R₆, R₇, R₈ and R₉ are as defined above for formula (II), e.g.,

wherein

a) R₆ is nitro, alkyl, substituted alkyl, phenyl, hydroxy, formyl, heteroalkylaryl, alkoxy, acyl or acyloxy, preferably alkyl, especially, C₁-C₇alkyl; hydroxyl or alkoxy, especially, C₁-C₇alkoxy; and
R₇, R₈ and R₉ are hydrogen; or

b) R₆, R₈ and R₉ are hydrogen; and
R₇ is alkyl, substituted alkyl, phenyl, halogen, alkoxy or cyano, preferably alkyl, especially, C₁-C₇alkyl; substituted alkyl, especially, substituted C₁-C₇alkyl, such as —CF₃; or alkoxy, especially, C₁-C₇alkoxy; or

c) R₆, R₇, R₉ are hydrogen; and
R₈ is alkyl, substituted alkyl, halogen, nitro, cyano, thioalkoxy, acyloxy, phenyl, alkylsulfonyl or carboxyalkyl, preferably alkyl, especially, C₁-C₇alkyl; substituted alkyl, especially, —CF₃; halogen or carboxyalkyl; or

d) R₆, R₇, R₈ are hydrogen; and
R₉ is alkyl, halogen or hydroxy; or

e) R₇ and R₉ are hydrogen; and
each of R₆ and R₈, independently, is halogen, alkyl, substituted alkyl, phenyl or cyano; or

f) each of R₇ and R₉ is alkyl or substituted alkyl; and
R₆ and R₆ are hydrogen; or

g) R₆ and R₉ are hydrogen;
R₇is alkyl or substituted alkyl; and
R₈ is nitro; or

h) R₈ and R₉ are hydrogen;
R₆ is cyano; and
R₇ is alkoxy; or

i) R₇ and R₈ are hydrogen;
R is alkyl, substituted alkyl, alkoxy or SCN; and
R₉ is alkyl or substituted alkyl; or

j) R₆ and R₇ are hydrogen;
R₆ is nitro or halogen; and
R₈ is alkyl or substituted alkyl; or

k) R₆, R₇, R₈ and R₉ are hydrogen; or

l) R₆ and R₇, together with the carbon atoms to which they are attached, form a phenyl group, preferably substituted with hydroxyl; and
R₈ and R₉ are hydrogen; or

m) R₆ and R₇ are hydrogen; and
R₈ and R₉, together with the carbon atoms to which they are attached, form a phenyl group; or

n) n is 0; or

o) n is 0; and
each of R₆, R₇, R₈ and R₉, independently, is hydrogen, alkyl or halogen and, more particularly, R₆, R₇, R₈ and R₉ are hydrogen; or

p) n is 0;
R₆, R₈ and R₉ are hydrogen; and
R₇ is alkyl; or

q) n is 0;
R₆, R₇and R₉ are hydrogen; and
R₈ is alkyl or halogen.

In another embodiment, A is of formula (Ie) and R₁ is of formula (II.2) 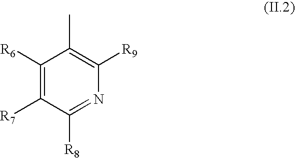
wherein R₆, R₇, R₈ and R₉ are as defined above for formula (II), in particular, R₇ and R₈, together with the carbon atoms to which they are attached, form a phenyl group and R₆ and R₉ are hydrogen.

In yet another embodiment, A is of formula (Ie) and R₁ is of formula (III) 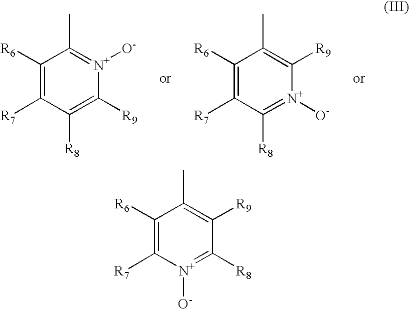
wherein each of R₆, R₇, R₈ and R₉, independently, is hydrogen, alkyl, substituted alkyl, phenyl, halogen, hydroxy or alkoxy, e.g.,
wherein

a) R₆ and R₈ are hydrogen;
R₉ is hydrogen or alkyl; and
R₇ is alkyl, substituted alkyl or phenyl; or

b) R₆, R₇ and R₉ are hydrogen; and
R₈ is halogen, alkyl or substituted alkyl; or

c) R₇, R₈ and R₉ are hydrogen; and
R₆ is hydroxy.

In a particularly useful embodiment, A is of formula (Ie) and R₁ is of the formula (III.1) 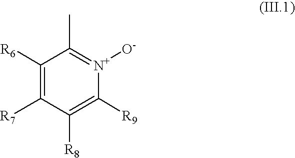
wherein R₆, R₇, R₈ and R₉ are as defined above for formula (III).

In another embodiment, R₁ is an unsubstituted phenyl or the phenyl is substituted with alkoxy, e.g., methoxy or aryloxy, e.g., phenoxy.

In another embodiment, the R₁ is of formula (IV) 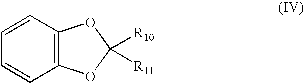
wherein each of R₁₀ and R₁₁, independently, is hydrogen or halogen. In particular, R₁₀ and R₁₁ are both either hydrogen or both halogen.

Unless otherwise stated, the following terms as used in the specification have the following meaning.

The term “side chain of a natural or a non-natural alpha-amino acid” is the group R^{x} in an amino acid of formula NH₂—CH(R^{x})—COOH. Examples of side chains of alpha-amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, alpha-aminoadipic acid, alpha-amino-n-butryic acid, 3,4,-dihydroxyphenylalanine, homoserine, alpha-methylserine, omithine, pipecolic acid, and thyroxine. In alpha-amino acid side chains which contain functional substituents, for example, amino, carboxyl, hydroxyl, mercapto, guanidyl, imidazolyl, or indolyl groups as in arginine, lysine, glutamic acid, aspartic acid, tryptophan, histidine, serine, threonine, tyrosine, and systeine, such functional substituents may optionally be protected.

The term “cycloalkane” or “cycloalkyl” contains from 3- to 7-ring carbon atoms and is, e.g., cyclopropyl, cyclobutyl, cyclopentyl and cyclohexyl.

The term “azacyclo4-7alkane” contains 1-ring heteroatom which is a nitrogen. It contains from 4-7 and, especially, 4- or 5-ring atoms including the heteroatoms.

The term “thiazacyclo4-7alkane” contains 2-ring heteroatoms, nitrogen and sulfur. It contains from 4-7 and, especially, 5-ring atoms including the heteroatoms.

The term “imidazacyclo₄₋₇alkane” contains 2-ring heteroatoms which are both nitrogen. It contains from 4-7 and, especially, 5-ring atoms including the heteroatoms.

The term “aliphatic group” refers to saturated or unsaturated aliphatic groups, such as alkyl, alkenyl or alkynyl, cycloalkyl or substituted alkyl including straight-chain, branched-chain and cyclic groups having from 1-10 carbons atoms. The term “alkyl” or “alk”, whenever it occurs, is a saturated straight chain or branched aliphatic group of 1-10 carbon atoms or a cycloalkyl of 3-10 carbon atoms, more preferably, alkyl groups are C₁-C₇alkyl, particularly, C₁-C₄alkyl. Examples of “alkyl” or “alk” include, but are not limited to, methyl, ethyl, n-propyl, isopropyl, n-butyl, isobutyl, sec-butyl, t-butyl, n-pentyl, neopentyl, n-hexyl or n-heptyl, cyclopropyl and, especially, n-butyl.

The term “substituted alkyl” refers to an alkyl group that is substituted with one or more substitutents, preferably, 1-3 substitutents including, but not limited to, substituents, such as halogen, lower alkoxy, hydroxy, mercapto, carboxy, cycloalkyl, aryl, heteroaryl and the like. Examples of substituted alkyl groups include, but are not limited to, —CF₃, —CF₂—CF₃, hydroxymethyl, 1- or 2-hydroxyethyl, methoxymethyl, 1- or 2-ethoxyethyl, carboxymethyl, 1- or 2-carboxyethyl and the like.

The term “aryl” or “Ar” refers to an aromatic carbocyclic group of 6-14 carbon atoms having a single ring including, but not limited to, groups, such as phenyl, or multiple condensed rings including, but not limited to, groups, such as naphthyl or anthryl; and is, especially, phenyl.

The term “heteroaryl” or “HetAr” refers to a 4- to 7-membered, monocyclic aromatic heterocycle or a bicycle that is composed of a 4- to 7-membered, monocyclic aromatic heterocycle and a fused-on benzene ring. The heteroaryl has at least one hetero atom, preferably, one or two heteroatoms including, but not limited to, heteroatoms, such as N, O and S, within the ring. A preferred heteroaryl group is pyridinyl, pyrimidinyl or benzdioxolanyl.

The aryl or heteroaryl may be substituted or unsubstituted by one or more substituents including, but not limited to, C₁-C₇alkyl, particularly, C₁-C₄alkyl, such as methyl, hydroxy, alkoxy, acyl, acyloxy, SCN, halogen, cyano, nitro, thioalkoxy, phenyl, heteroalkylaryl, alkylsulfonyl and formyl.

The term “heterocyclic” includes “heteroaryl” as defined above, and, in particular means a 5-7 membered aromatic or non-aromatic heterocyclic ring containing one or more heteroatoms selected from S, N and O, and optionally fused to a benzene ring, including for example, pyrrolyl, furyl, thienyl, piperidinyl, imidazolyl, oxazolyl, thiazolyl, thiadiazolyl, pyrazolyl, pyridinyl, pyrrolidinyl, pyrimidinyl, morpholinyl, piperazinyl, indolyl, benzimidazolyl, maleimido, succinimido, phthalimido and 1,3-dioxo-1,3-dihydro-isoindol-2-yl groups.

The term “carbonylamine”, as used herein, refers to a —NHC(O)— group, wherein the amino portion of the group is linked to the aryl/heteroaryl and the carbonyl portion of the group is linked to the azacyclo₄₋₇alkane, thiazacyclo₄₋₇alkane or imidazacyclo₄₋₇alkane.

The term “heteroalkyl” refers to saturated or unsaturated C₁-C₁₀alkyl as defined above and, especially, C₁-C₄heteroalkyl, which contain one or more heteroatoms, as part of the main, branched or cyclic chains in the group. Heteroatoms may independently be selected from the group consisting of —NR—, where R is hydrogen or alkyl, 13 S—, —O— and —P—, preferably, —NR—, where R is hydrogen or alkyl and/or —O—. Heteroalkyl groups may be attached to the remainder of the molecule either at a heteroatom (if a valence is available) or at a carbon atom. Examples of heteroalkyl groups include, but are not limited to, groups, such as —O—CH₃, —CH₂—O—CH₃, —CH₂—CH₂O—CH₃, —S—CH₂—CH₂—CH₃, —CH₂—CH(CH₃)—S—CH₃ and —CH₂13 CH₂NH—CH₂—CH₂—.

The heteroalkyl group may be substituted or unsubstituted with one or more substituents, preferably, 1-3 substituents including, but not limited to, alkyl, halogen, alkoxy, hydroxyl, mercapto, carboxy and, especially, phenyl. The heteroatom(s), as well as the carbon atoms of the group, may be substituted. The heteroatom(s) may also be in oxidized form.

The term “alkoxy”, as used herein, refers to a C₁-C₁₀alkyl linked to an oxygen atom or, preferably, C₁-C₇alkoxy, more preferably, C₁-C₄alkoxy. Examples of alkoxy groups include, but are not limited to, groups, such as methoxy, ethoxy, n-butoxy, tert-butoxy and allyloxy.

The term “acyl”, as used herein, refers to the group —(O)CR, where R is alkyl, especially, C₁-C₇alkyl, such as methyl. Examples of acyl groups include, but are not limited to, acetyl, propanoyl and butanoyl.

The term “acyloxy”, as used herein, refers to the group —OC(O)R, wherein R is hydrogen, alkyl, especially, C₁-C₇alkyl, such as methyl or ethyl, or phenyl or substituted alkyl as defined above.

The term “alkoxycarbonyl”, as used herein, refers to the group —COOR, wherein R is alkyl, especially, C₁-C₇alkyl, such as methyl or ethyl.

The term “halogen” or “halo”, as used herein, refers to chlorine, bromine, fluorine, iodine and, especially, fluorine.

The term “thioalkoxy”, as used herein, means a group —SR, where R is an alkyl as defined above, e.g., methylthio, ethylthio, propylthio, butylthio and the like.

The term “heteroalkylaryl”, as used herein, means a heteroalkyl group, e.g., —O—CH₂— substituted with an aryl group, especially, phenyl. The phenyl group itself may also be substituted with one or more substituents, such as halogen, especially, fluoro and chloro, and alkoxy, such as methoxy.

The term “alkylsulfonyl”, as used herein, means a group —SO₂R, wherein R is alkyl, especially, C₁-C₇alkyl, such as methyl sulfonyl. “Protecting group” refers to a chemical group that exhibits the following characteristics:

1) reacts selectively with the desired functionality in good yield to give a protected substrate that is stable to the projected reactions for which protection is desired;

2) is selectively removable from the protected substrate to yield the desired functionality; and

3) is removable in good yield by reagents compatible with the other functional group(s) present or generated in such projected reactions.

Examples of suitable protecting groups may be found in Greene et al., “Protective Groups in Organic Synthesis”, 2^{nd} Edition, John Wiley & Sons, Inc., NY (1991). Preferred amino protecting groups include, but are not limited to, benzyloxycarbonyl (CBz), t-butyl-oxycarbonyl (Boc), t-butyidimethylsilyl (TBDMS), 9-fluorenylmethyl-oxycarbonyl (Fmoc) or suitable photolabile protecting groups, such as 6-nitroveratryloxy carbonyl (Nvoc), nitropiperonyl, pyrenylmethoxycarbonyl, nitrobenzyl, dimethyl dimethoxybenzyl, 5-bromo-7-nitroindolinyl and the like. Preferred hydroxy protecting groups include Fmoc, TBDMS, photolabile protecting groups, such as nitroveratryl oxymethyl ether (Nvom), methoxy methyl ether (Mom), and methoxy ethoxy methyl ether (Mem). Particularly preferred protecting groups include 4-nitrophenethyloxycarbonyl (NPEOC) and 4-nitrophenethyloxymethyloxycarbonyl (NPEOM).

It will be appreciated that the compounds of formula (I) may exist in the form of optical isomers, racemates or diastereoisomers. For example, a compound of formula (I), wherein R₂ and R₃ are different residues; or wherein R₄ and R₅ are different residues, is asymmetric and may have the R- or S-configuration. It is to be understood that the present invention embraces all enantiomers and their mixtures. Similar considerations apply in relation to starting materials exhibiting asymetric carbon atoms as mentioned.

In the compounds of formula (I), the following significances are preferred individually or in any sub-combination:

1. A is of formula (Ie).

2. R₁ is a heteroaryl of formula (II.1),

wherein
R₆, R₇ and R₉ are hydrogen; and
R₈ is methyl or trifluoromethyl; or
R₆, R₇ and R₈ are hydrogen; and
R₉ is fluoro; or
R₆, R₈ and R₉ are hydrogen; and
R₇ is ethyl or methoxy; or
R₇, R₈ and R₉ are hydrogen; and
R₆ is hydroxy; or
R₇ and R₈ are hydrogen;
R₆ is methoxy; and
R₉ is methyl; or

R₁ is a heteroaryl of formula (III.1),

wherein
R₆, R₇ and R₉ are hydrogen; and
R₆ is fluoro or trifluoromethyl; or
R₆, R₈ and R₉ are hydrogen; and R₇ is ethyl; preferably,

R₁ is a heteroaryl of formula (II.1),

wherein
R₆, R₈ and R₉ are hydrogen; and
R₇ is ethyl or a heteroaryl of formula (III.1),
wherein
   R₆, R₇ and R₉ are hydrogen; and
   R₆ is fluoro.
      3. X is —CH₂—, —CH(OH)—, —CH(OR)—, —CF₂— or —CH(F)—, preferably, X is —CH₂—;
      4. R₂, R₃, R₄ are hydrogen;
      5. R₆ is alkyl, preferably, C₁-C₇alkyl, such as n-butyl;
      6. n is 1.

The compounds disclosed herein of formula (I) which are used to prepare crystalline salts of the invention can be prepared by the processes disclosed in WO 02/102790 A1 and WO 99/39704.

The crystalline salt compounds of the present invention are, therefore, useful for the treatment and/or prevention of infectious disorders caused by a variety of bacterial or prokaryotic organisms. Examples include, but are not limited to, Gram positive and Gram negative aerobic and anaerobic bacteria including Staphylococci, e.g., S. aureus and S. epidermidis; Enterococci, e.g., E. faecalis and E. faecium; Streptococci, e.g., S. pneumoniae; Haemophilus, e.g., H. influenza; Moraxella, e.g., M. catarrtalis; and Escherichia, e.g., E. coli. Other examples include Mycobacteria, e.g., M. tuberculosis; intercellular microbes, e.g., Chlamydia and Rickettsiae; and Mycoplasma, e.g., M. pneumoniae; and Pseudomonas, e.g., P. aeruginosa; H. pylor; and parasites, e.g., Plasmodium falciparum.

As used herein, an “infectious disorder” is any disorder characterized by the presence of a microbial infection, such as the presence of bacteria. Such infectious disorders include, e.g., central nervous system infections; external ear infections; infections of the middle ear, such as acute otitis media; infections of the cranial sinuses; eye infections; infections of the oral cavity, such as infections of the teeth, gums and mucosa; upper respiratory tract infections; lower respiratory tract infections; genitourinary infections; gastrointestinal infections; gynecological infections; septicemia; bone and joint infections; skin and skin structure infections; bacterial endocarditis; bums; antibacterial prophylaxis of surgery; antibacterial prophylaxis in immunosuppressed patients, such as patients receiving cancer chemotherapy, or organ transplant patients and chronic diseases caused by infectious organisms, e.g., arteriosclerosis.

The crystalline salt compounds of the invention may be used to treat a subject to treat, prevent and/or reduce the severity of an infection. Subjects include animals, plants, blood products, cultures and surfaces, such as those of medical or research equipment, such as glass, needles, surgical equipment and tubing, and objects intended for temporary or permanent implantation into an organism. Preferred animals include mammals, e.g., mice, rats, cats, dogs, cows, sheep, pigs, horses, swine, primates, such as rhesus monkeys, chimpanzees, gorillas and, most preferably, humans. Treating a subject includes, but is not limited to, preventing, reducing and/or eliminating the clinical symptoms caused by an infection of a subject by a microorganism; preventing, reducing and/or eliminating an infection of a subject by a microorganism; or preventing, reducing and/or eliminating contamination of a subject by a microorganism. The microorganism involved is preferably a prokaryote, more preferably, a bacterium.

For the above uses the required dosage will of course vary depending on the mode of administration, the particular condition to be treated and the effect desired. The compositions may contain, e.g., from about 0.1% by weight to about 99% by weight, e.g., from about 10-60% by weight, of the active material, depending on the method of administration. Where the compositions comprise dosage units, each unit will contain, e.g., from about 1-1000 mg, e.g., 1-500 mg, of the active ingredient. The dosage as employed for adult human treatment will range, e.g., from about 1-3000 mg/day, for instance, 1500 mg/day depending on the route and frequency of administration. Such a dosage corresponds to about 0.015-50 mg/kg/day. Suitably the dosage is, e.g., from about 5-20 mg/kg/day. Suitable unit dosage forms for oral administration comprise ca. 0.25-1500 mg active ingredient.

A “pharmaceutically acceptable carrier” means an excipient that is useful in preparing a pharmaceutical composition that is generally safe, non-toxic and neither biologically nor otherwise undesirable, and includes an excipient that is acceptable for veterinary use, as well as human pharmaceutical use. A “pharmaceutically acceptable carrier” as used in the specification and claims includes both one and more than one such carriers.

The crystalline salt compounds of the invention may be administered by any conventional route, e.g., locally or systemically, e.g., orally, topically, parenterally, subdermally or by inhalation and may be used for the treatment of bacterial infection in a subject, such as animals, preferably, mammals and, more preferably, humans.

The crystalline salt compounds of the invention may be formulated for administration in any convenient way for use in human or veterinary medicine, by analogy with other antibiotics. Such methods are known in the art (see, e.g., Remington's Pharmaceutical Sciences, Mack Publishing Co., Easton, Pa.) and are not described in detail herein.

The compositions may be in any form known in the art including, but not limited to, tablets, capsules, wafers, fast melts (without wafers), powders, granules, lozenges, creams or liquid preparations, such as oral or sterile parenteral solutions or suspensions. The compounds may also be administered in liposomal, micellar or microemulsion formulations. The compounds may also be administered as prodrugs, where the prodrug administered undergoes biotransformation in the treated mammal to a form which is biologically active.

The topical formulations of the present invention may be presented as, for instance, ointments, creams or lotions, solutions, salves, emulsions, plasters, eye ointments and eye or ear drops, impregnated dressings, transdermal patches, sprays and aerosols, and may contain appropriate conventional additives, such as preservatives, solvents to assist drug penetration and emollients in ointments and creams.

The formulations may also contain compatible conventional carriers, such as cream or ointment bases and ethanol or oleyl alcohol for lotions. Such carriers may be present, e.g., from about 1% up to about 99% of the formulation. For example, they may form up to about 80% of the formulation.

Tablets and capsules for oral administration may be in unit dose presentation form, and may contain conventional excipients, such as binding agents, e.g., syrup, acacia, gelatin, sorbitol, tragacanth or polyvinylpyrollidone; fillers, e.g., lactose, sugar, maize-starch, calcium phosphate, sorbitol or glycine; tabletting lubricants, e.g., magnesium stearate, talce polyethylene glycol or silica; disintegrants, e.g., potato starch; or acceptable wetting agents, such as sodium lauryl sulphate. The tablets may be coated according to methods well-known in standard pharmaceutical practice.

Oral liquid preparations may be in the form of, e.g., aqueous or oily suspensions, solutions, emulsions, syrups or elixirs, or may be presented as a dry product for reconstitution with water or another suitable vehicle before use. Such liquid preparations may contain conventional additives, such as suspending agents, e.g., sorbitol, methyl cellulose, glucose syrup, gelatin, hydroxyethyl cellulose, carboxymethyl cellulose, aluminum stearate gel or hydrogenated edible fats; emulsifying agents, e.g., lecithin, sorbitan monooleate or acacia; non-aqueous vehicles, which may include edible oils, e.g., almond oil, oily esters, such as glycerine, propylene glycol or ethyl alcohol; preservatives, e.g., methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, may be either suspended or dissolved in the vehicle or other suitable solvent. In preparing solutions, the compound may be dissolved in water for injection and filter sterilized before filling into a suitable vial or ampule and sealing. Advantageously, agents, such as a local anesthetic preservative and buffering agents may be dissolved in the vehicle. To enhance the stability, the composition may be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound may be sterilized by exposure to ethylene oxide before suspending in the sterile vehicle. Advantageously, a surfactant or wetting agent is included in the composition to facilitate uniform distribution of the compound.

In accordance with the foregoing the present invention further provides:

1.1. A method for treating and/or preventing an infectious disorder in a subject, such as a human or other animal subject, comprising administering to the subject an effective amount of a crystalline salt compound of the invention, e.g., of formula (I), or a prodrug thereof.

1.2. A method for inhibiting PDF in a subject comprising administering to the subject an effective PDF inhibiting amount of a crystalline salt compound of the invention, e.g., of formula (I), or a prodrug thereof.

1.3. A pharmaceutical composition, e.g., for use in any of the methods, as in 1.1 or 1.2 above, comprising a compound of the invention, e.g., a crystalline salt of formula (I), in association with a pharmaceutically acceptable diluent or carrier therefor.

1.4. A compound of the invention, e.g., crystalline salt of formula (I), or a prodrug thereof for use as a pharmaceutical or in the preparation of a pharmaceutical composition for use in any method as indicated under 1.1 or 1.2 above.

“Treating” or “treatment” of a disease includes:

(1) preventing the disease, i.e., causing the clinical symptoms of the disease not to develop in a subject, e.g., a mammal, that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

An “effective PDF inhibiting amount” means the amount of a compound or a prodrug thereof, that when administered to a subject for treating an infectious disorder responsive to inhibition of PDF or for inhibiting PDF, is sufficient to inhibit PDF. The “effective PDF inhibiting amount” will vary depending on the compound, salt thereof or prodrug thereof, employed, the microorganism that is inhibited in the subject, the age, weight, sex, medical condition, species, disorder and its severity, of the subject to be treated and the route of administration, but may nevertheless be readily determined by one skilled in the art.

The compounds of the invention, e.g., crystalline salts of formula (I), or prodrug thereof, may be administered alone or in combination with another therapeutic agent. Examples of such therapeutic agents include, but are not limited to, other antibacterial agents, such as β-lactams, e.g., penicillins; cephalosporins; carbapenems; ketolides; quinolones, e.g., fluoroquinolones; macrolides, e.g., clarithromycin, azithromycin or vancomycin; rifamycins; monobactams; isoniazid; licosamides; mupirocin; sulfonamides; phenicols; fosfomycin; glycopeptides; tetracyclines; streptogramins; chloramphenicol; and oxazolidinone, anti-inflammatory agents, e.g., corticosteroids or NSAID, analgesics, e.g., narcotic or non-opioic analgesics.

In accordance with the foregoing, the present invention provides in a yet further aspect:

1.5. A method as defined above comprising co-administration, e.g., concomitantly or in sequence, of a therapeutically effective amount of a compound of the invention, e.g., a crystalline salt of formula (I), or a prodrug thereof, and a second therapeutic agent.

1.6. A therapeutic combination, e.g., a kit, comprising:
a) a compound of the invention, e.g., a crystalline salt of formula (I) or a prodrug thereof; and
b) at least one second therapeutic agent.

Component a) and component b) may be used concomitantly or in sequence. The kit may comprise instructions for its administration.

The following are representative pharmaceutical formulations containing a compound of formula (I).

Tablet Formulation

The following ingredients are mixed intimately and pressed into single scored tablets:

| Quantity per Ingredient | Tablet (mg) |
|---|---|
| Compound of this invention | 400 |
| Cornstarch | 50 |
| Croscarmellose sodium | 25 |
| Lactose | 120 |
| Magnesium stearate | 5 |

Capsule Formulation

The following ingredients are mixed intimately and loaded into a hard-shell gelatin capsule:

| Quantity per Ingredient | Ingredient Capsule (mg) |
|---|---|
| Compound of this invention | 200 |
| Lactose, spray -- dried | 148 |
| Magnesium stearate | 2 |

Suspension Formulation

The following ingredients are mixed to form a suspension for oral administration:

| Ingredient | Amount | |
|---|---|---|
| Compound of this invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.00 | g |
| Veegum K (Vanderbilt Co.) | 1.0 | g |
| Flavoring | 0.035 | mL |
| Colorings | 0.5 | mg |
| Distilled water | q.s. to 100 | mL |

Injectable Formulation

The following ingredients are mixed to form an injectable formulation:

| Ingredient | Amount | |
|---|---|---|
| Compound of this invention | 0.2-20 | mg |
| Sodium acetate buffer solution, 0.4 M | 20 | ml |
| HCl (1 N) or NaOH (1 N) | q.s. to suitable pH | |
| Water (distilled, sterile) | q.s. to 20 | mL |

Suppository Formulation

A suppository of total weight 2.5 g is prepared by mixing the compound of the invention with Witepsol® H-5 (triglycerides of saturated vegetable fatty acid; Riches-Nelson, Inc., NY) and has the following composition:

| | |
|---|---|
| Compound of the invention | 500 mg |
| Witepsol ® H-15 | 2000 mg |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

### EXAMPLE 1

### Preparation of the calcium crystalline salt of 1-{2-R-[(formyl-hydroxy-amino)-methyl]-hexanoyl}-pyrrolidine-2-S-carboxylic acid-(4-ethyl-pyridin-2-yl)-amide

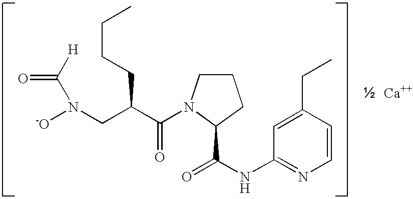

A mixture of 30 mg of amorphous 1-{2-R-[(formyl-hydroxy-amino)-methyl]-hexanoyl}-pyrrolidine-2-S-carboxylic acid-(4-ethyl-pyridin-2-yl)-amide in 0.3 mL of absolute ethanol and 0.5 mL of water is stirred at room temperature (“RT”). To this is added 12.6 μL of 6 M aqueous sodium hydroxide followed by 18.9 μL of 2 M aqueous calcium chloride solution. The mixture is stirred for 24 hours at RT and the resulting solid is isolated by filtration and dried at 20° C. under vacuum. The crystallinity is determined by X-ray powder diffraction chrystallography.

### EXAMPLE 2

### Preparation of the crystalline magnesium salt of 1-{2-R-[(formyl-hydroxy-amino)-methyl]-hexanoyl}-pyrrolidine-2-S-carboxylic acid (5-fluoro-1-oxy-pyridin-2-yl)-amide

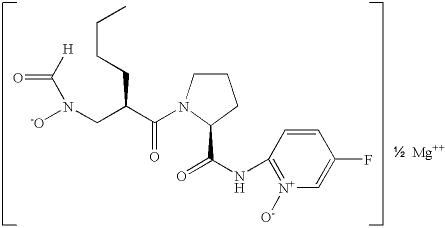

A mixture of 30 mg of amorphous 1-{2-R-[(formyl-hydroxy-amino)-methyl]-hexanoyl}-pyrrolidine-2-S-carboxylic acid (5-fluoro-1-oxy-pyridin-2-yl)-amide in 0.6 mL of water is stirred for 10 minutes at 20° C. to effect solution. To this is added 15.1 μL of 5 M aqueous sodium hydroxide and the solution was stirred for 5 minutes. Subsequently, 18.9 μL of 2 M aqueous magnesium chloride solution is added. The mixture is stirred for 1.5 hours at RT and the resulting solid is isolated by filtration and dried at 40° C. under vacuum. The crystallinity is determined by X-ray powder diffraction chrystallography.

A typical X-ray powder diffraction pattern (Cu K_{α} radiation) for the compound of Example 2 at 50% relative humidity is as follows:

| 2-Theta (Deg.) | D (Angström*) | CPS | Relative Intensity |
|---|---|---|---|
| 6.8475 | 11.6562 | 2707.63 | 100 |
| 12.7025 | 6.2926 | 454.9 | 16.8 |
| 13.0337 | 6.1333 | 342.82 | 12.66 |
| 13.675 | 5.847 | 1764.03 | 65.15 |
| 14.195 | 5.6339 | 417.33 | 15.41 |
| 14.535 | 5.5028 | 719.98 | 26.59 |
| 15.225 | 5.2547 | 394.27 | 14.56 |
| 15.48 | 5.1687 | 177.5 | 6.56 |
| 18.1175 | 4.4212 | 2229.28 | 82.33 |
| 19.7744 | 4.054 | 193.18 | 7.13 |
| 20.5837 | 3.8962 | 1550 | 57.25 |
| 21.9169 | 3.6619 | 424.53 | 15.68 |
| 22.4031 | 3.5834 | 425.17 | 15.7 |
| 23.54 | 3.4126 | 212.5 | 7.85 |
| 23.72 | 3.387 | 186.92 | 6.9 |
| 24.2887 | 3.3089 | 302.38 | 11.17 |
| 24.4656 | 3.2853 | 404.03 | 14.92 |
| 25.0056 | 3.2155 | 300.8 | 11.11 |
| 25.925 | 3.1033 | 343.42 | 12.68 |
| 29.32 | 2.7505 | 283.67 | 10.48 |
| 30.8019 | 2.6212 | 413.8 | 15.28 |
| 32.5206 | 2.4861 | 253.8 | 9.37 |

*1 Angström = 10⁻¹⁰ m

### EXAMPLE 3

### Preparation of the crystalline zinc salt of 1-{2-R-[(formyl-hydroxy-amino)-methyl]-hexanoyl}-pyrrolidine-2-S-carboxylic acid (5-fluoro-1-oxy-pyridin-2-yl)-amide

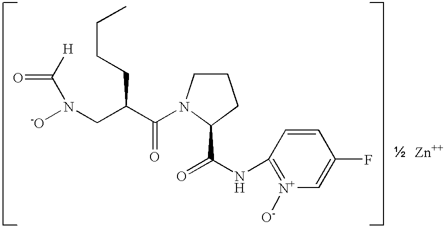

A mixture of 30 mg of amorphous 1-{2-R-[(formyl-hydroxy-amino)-methyl]-hexanoyl}-pyrrolidine-2-S-carboxylic acid (5-fluoro-1-oxy-pyridin-2-yl)-amide in 0.6 mL of water is stirred for 10 minutes at 20° C. to effect solution. To this is added 15.1 μL of 5 M aqueous sodium hydroxide and the resulting solution was stirred for 5 minutes. Subsequently, 99.6 μL of a 0.38 M aqueous zinc sulfate solution is added. The mixture is stirred for 10 minutes at RT and the resulting solid is isolated by filtration and dried at 40° C. under vacuum for 15 hours. The crystallinity is determined by X-ray powder diffraction chrystallography.

### EXAMPLE 4

### Preparation of the crystalline calcium salt of 1-{2-R-[(formyl-hydroxy-amino)-methyl]-hexanoyl}-pyrrolidine-2-S-carboxylic acid (5-fluoro-1-oxy-pyridin-2-yl)-amide

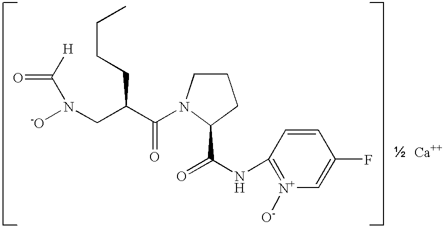

A mixture of 126.9 mg of amorphous 1-{2-R-[(formyl-hydroxy-amino)-methyl]-hexanoyl}pyrrolidine-2-S-carboxylic acid (5-fluoro-1-oxy-pyridin-2-yl)-amide in 2.5 mL of water is stirred for 10 minutes at 20° C. to effect solution. To this is added 0.064 mL of 5 M aqueous sodium hydroxide and the solution was stirred for 5 minutes. Subsequently, a 0.6 mL aliquot is removed and treated with 0.056 mL of a 0.68 M aqueous solution of calcium chloride. The mixture is stirred overnight at RT and the resulting solid is isolated by filtration and dried at 40° C. for 5 hours under vacuum and then 3 days at RT under vacuum. The dried material is left in contact with air for several hours prior to analysis. The crystallinity is determined by X-ray powder diffraction chrystallography.

### EXAMPLE 5

### Preparation of the calcium crystalline salt of 2R-[(formyl-hydroxy-amino)-methyl]-hexanoic acid (1S-dimethylcarbamoyl-2,2-dimethyl-propyl)-amide

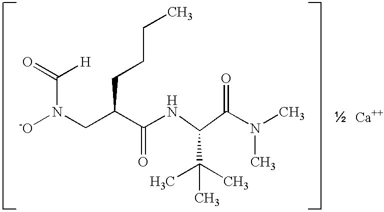

A mixture of 30 mg of amorphous 2R-[(formyl-hydroxy-amino)-methyl]-hexanoic acid (1S-dimethylcarbamoyl-2,2-dimethyl-propyl)-amide in 0.6 mL of water is stirred at RT. To this is added 15.2 μL of 6 M aqueous sodium hydroxide followed by 22.8 μL of 2 M aqueous calcium chloride solution. The mixture is warmed to 40° C. and then stirred at 20° C. for 24 hours at RT. The resulting solid is isolated by filtration and dried at 20° C. under vacuum. The crystallinity is determined by X-ray powder diffraction chrystallography.

### EXAMPLE 6

### Preparation of the calcium crystalline salt of N-(2,2-diphenylethyl)-2-[(formylhydroxyamino)methyl]-(2R)-hexanamide

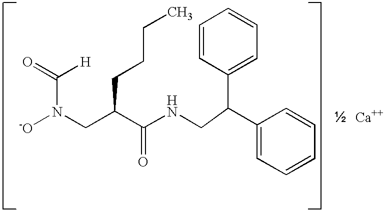

A mixture of 30 mg of amorphous N-(2,2-diphenylethyl)-2-[(formylhydroxyamino)methyl]-(2R)hexanamide in 0.4 mL of absolute ethanol and 0.6 mL of water is stirred at RT. To this is added 13.6 μL of 6 M aqueous sodium hydroxide followed by 20.4 μL of 2 M aqueous calcium chloride solution. The mixture is stirred for 24 hours at RT and the resulting solid is isolated by filtration and dried at 20° C. under vacuum. The crystallinity is determined by X-ray powder diffraction chrystallography.

### EXAMPLE 7

### Preparation of the sodium crystalline salt of N-(2,2-diphenylethyl)-2-[(formylhydroxyamino)methyl]-(2R)-hexanamide

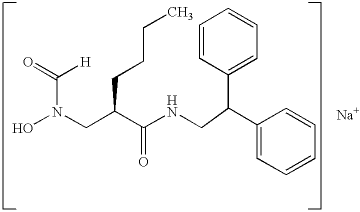

A mixture of 30 mg of amorphous N-(2,2-diphenylethyl)2-[(formylhydroxyamino)methyl]-(2R)-hexanamide in 0.4 mL of absolute ethanol and 0.6 mL of water is stirred at RT. To this is added 13.6 μL of 6 M aqueous sodium hydroxide solution. The mixture is stirred at 40° C. for 1 hour and then at 20° C. for 24 hours. The resulting solid is isolated by evaporating the solvent at 20° C. under vacuum and drying for 16 hours. The crystallinity is determined by X-ray powder diffraction chrystallography.

1. A crystalline salt of formula (1): 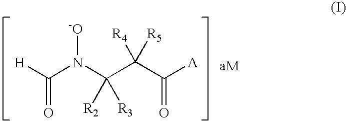
wherein
M is a mono- or di-valent metal;
a is ½ or 1;
each of R₂, R₃, R₄ and R₅, independently, is hydrogen or an aliphatic group, or (R₂ or R₃) and (R₄ or R₅), collectively, form a C₄-C₇cycloalkyl;
A is of the formula (Ia), (Ib), (Ic), (Id) or (Ie) 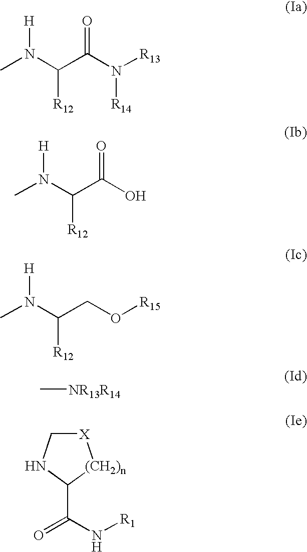
wherein
R₁₂ is the side-chain of a natural or a non-natural alpha amino acid;
R₁₃ and R₁₄, independently, represent hydrogen, or optionally substituted C₁-C₈alkyl, cycloalkyl, aryl, aryl( C₁-C₆alkyl), heterocyclic or heterocyclic( C₁-C₆alkyl);
R₁₅ is hydrogen, C₁-C₆alkyl or an acyl group;
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl; and
n is 0-3, provided that when n is 0, X is —CH₂—.

2. The crystalline salt of claim 1, wherein A is formula (Ie).

3. The crystalline salt of claim 2,
wherein
a is ½; and
M is Ca, Zn or Mg.

4. The crystalline salt of claim 2,
wherein
A is of formula (Ie); and
R₁ is a heteroaryl of formula (II.1) 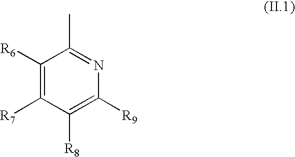
wherein
R₆, R₇ and R₉ are hydrogen; and
R₈ is methyl or trifluoromethyl; or
R₆, R₇ and R₈ are hydrogen; and
R₉ is fluoro; or
R₆, R₈ and R₉ are hydrogen; and
R₇ is ethyl or methoxy; or
R₇, R₈ and R₉ are hydrogen; and
R₆ is hydroxy; or
R₇ and R₈ are hydrogen;
R₆ is methoxy; and
R₉ is methyl.

5. The crystalline salt of claim 4,
wherein
R₆, R₈ and R₉ are hydrogen; and
R₇ is ethyl.

6. The crystalline salt of claim 2,
wherein
A is of formula (Ie); and
R₁ is of the formula (III.1) 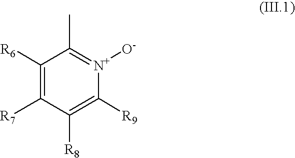
wherein
R₆, R₇ and R₉ are hydrogen; and
R₈ is fluoro or trifluoromethyl; or
R₆, R₈ and R₉ are hydrogen; and
R₇ is ethyl.

7. The crystalline salt of claim 6,
wherein
R₆, R₇ and R₉ are hydrogen; and
R₈ is fluoro.

8. The crystalline salt of claim 7,
wherein
a is ½; and
M is Ca, Zn or Mg.

9. The crystalline salt of claim 1, containing at least 2% water.

10. The crystalline salt of claim 1, containing about 8% water to about 9% water.

11. The crystalline salt of claim 1, wherein the X-ray powder diffraction pattern comprises crystalline peaks with 2-theta angles (Cu—K_{α} radiation) at least five of the following positions:
6.8±0.1, 13.7±0.1, 12.2±0.1, 14.5±0.1, 15.2±0.1, 18.1±0.1, 20.6±0.1, 22.0±0.1, 22.4±0.1, 24.5±0.1 and 30.9±0.1.

12. A hydrated crystalline magnesium salt of 1-{2-R-[(formyl-hydroxy-amino)-methyl]-hexanoyl}-pyrrolidine-2-S-carboxylic acid (5-fluoro-1-oxy- pyridin-2-yl)-amide, in particular a corresponding tetrahydrate salt.

13. A process for preparing a crystalline salt of the formula (I) 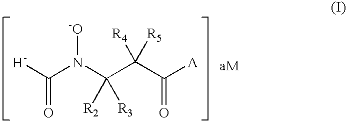
wherein
M is a mono- or di-valent metal;
a is ½ or 1;
each of R₂, R₃, R₄ and R₅, independently, is hydrogen or an aliphatic group, or (R₂ or R₃) and (R₄ or R₅), collectively, form a C₄-C₇cycloalkyl;
A is of the formula (Ia), (Ib), (Ic), (Id) or (Ie) 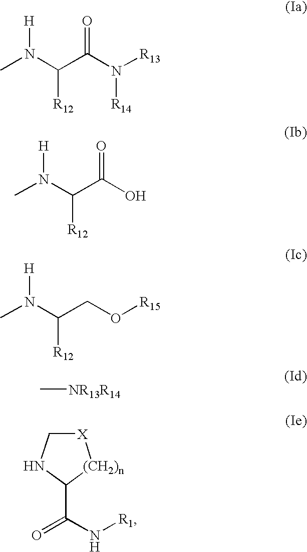
wherein
R₁₂ is the side-chain of a natural or a non-natural alpha amino acid;
R₁₃ and R₁₄, independently, represent hydrogen, or optionally substituted C₁-C₈alkyl, cycloalkyl, aryl, aryl( C₁-C₆alkyl), heterocyclic or heterocyclic(C₁-C₆alkyl);
R₁₅ is hydrogen, C₁-C₆alkyl or an acyl group;
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl; and
n is 0-3, provided that when n is 0, X is —CH₂—;
comprising dissolving the amorphous, non-salt form of the compound of formula (I) in a suitable solvent, contacting the dissolved compound with a base and with a metal salt, under conditions suitable to form the desired crystalline salt of formula (I).

14. A method for treating and/or preventing an infectious disorder in a subject, comprising administering to the subject an effective amount of a crystalline salt of formula (I): 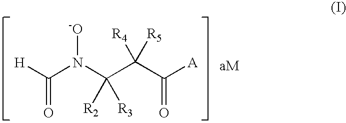
wherein
M is a mono- or di-valent metal;
a is ½ or 1;
each of R₂, R₃, R₄ and R₅, independently, is hydrogen or an aliphatic group, or (R₂ or R₃) and (R₄ or R₅), collectively, form a C₄-C₇cycloalkyl;
A is of the formula (Ia), (Ib), (Ic), (Id) or (Ie) 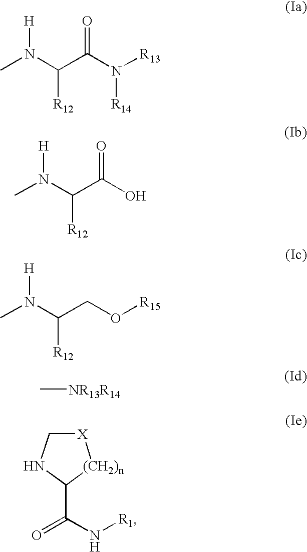
wherein
R₁₂ is the side-chain of a natural or a non-natural alpha amino acid;
R₁₃ and R₁₄, independently, represent hydrogen, or optionally substituted C₁-C₈alkyl, cycloalkyl, aryl, aryl( C₁-C₆alkyl), heterocyclic or heterocyclic(C₁-C₆alkyl);
R₁₅ is hydrogen, C₁-C₆alkyl or an acyl group;
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl; and
n is 0-3, provided that when n is 0, X is —CH₂—;
or a prodrug thereof.

15. The method of claim 14, comprising co-administration of a therapeutically effective amount of the crystalline salt of formula (I), or a prodrug thereof, and a second therapeutic agent.

16. A pharmaceutical composition comprising a crystalline salt of formula (I), 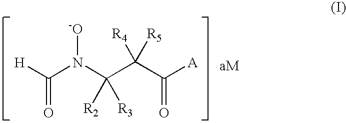
wherein
M is a mono- or di-valent metal;
a is ½ or 1;
each of R₂, R₃, R₄ and R₅, independently, is hydrogen or an aliphatic group, or (R₂ or R₃) and (R₄ or R₅), collectively, form a C₄-C₇cycloalkyl;
A is of the formula (Ia), (Ib), (Ic), (Id) or (Ie) 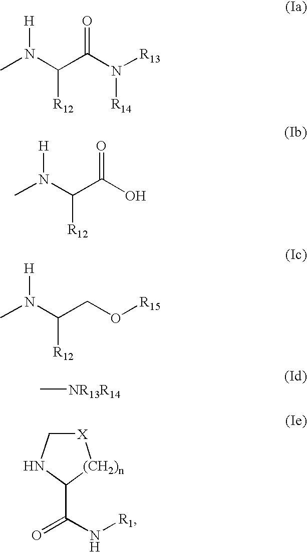
wherein
R₁₂ is the side chain of a natural or a non-natural alpha amino acid;
R₁₃ and R₁₄, independently, represent hydrogen, or optionally substituted C₁-C₈alkyl, cycloalkyl, aryl, aryl(C₁-C₆alkyl), heterocyclic or heterocyclic( C₁-C₆ alkyl);
R₁₅ is hydrogen, C₁-C₆alkyl or an acyl group;
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl; and
n is 0-3, provided that when n is 0, X is —CH₂—;
or a prodrug thereof,
in association with a pharmaceutically acceptable diluent or carrier therefor.

17. A composition according to claim 16 further comprising a second therapeutic agent.

18. Use of a crystalline salt of formula (I): 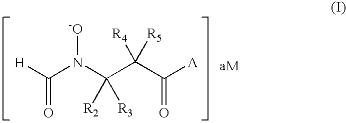
wherein
M is a mono- or di-valent metal;
a is ½ or 1;
each of R₂, R₃, R₄ and R₅, independently, is hydrogen or an aliphatic group, or (R₂ or R₃) and (R₄ or R₅), collectively, form a C₄-C₇cycloalkyl;
A is of the formula (Ia), (Ib), (Ic), (Id) or (Ie) 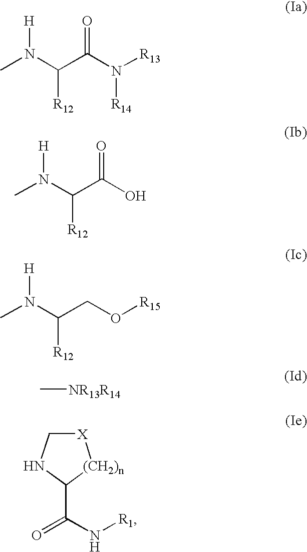
wherein
R₁₂ is the side-chain of a natural or a non-natural alpha amino acid;
R₁₃ and R₁₄, independently, represent hydrogen, or optionally substituted C₁-C₈alkyl, cycloalkyl, aryl, aryl(C₁-C₆alkyl), heterocyclic or heterocyclic(C₁-C₆alkyl);
R₁₅ is hydrogen, C₁-C₆alkyl or an acyl group;
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl; and
n is 0-3, provided that when n is 0, X is —CH₂—;
or a prodrug thereof, optionally together with a second therapeutical agent, in the manufacture of a medicament method for treating and/or preventing an infectious disorder.

19. The crystalline salt of claim 5,
wherein
a is ½; and
M is Ca, Zn or Mg.
